(19) United States
(12) Patent Application Publication
(10) Pub. No.: US 2023/0145153 A1
(43) Pub. Date: May 11, 2023

(54) Cool Pack

(71) Applicant: Calvin Doyle, Sandwich, MA (US)

(72) Inventor: Calvin Doyle, Sandwich, MA (US)

(21) Appl. No.: 17/520,874

(22) Filed: Nov. 8, 2021

(51) Int. Cl. F25B 21/04; A61F 7/02

(52) U.S. Cl. CPC F25B 21/04; A61F 7/02; A61F 2007/0024

(57) ABSTRACT

A device having a temperature control function of a new structure to adjust the body temperature according to the weather or temperature conditions. The device may comprising a device body having an article storage portion therein and a shoulder strap suspended from the device body, the face of the wearer of the device body The backrest may be provided with a heat dissipation member that is selectively heated or cooled by the thermoelectric module, the device body a battery unit for applying power to the thermoelectric module and a body temperature control function provided with an operation unit having switches and configured to turn on or off the temperature of the thermoelectric module, or control the temperature, or switch to a cooling mode or a heating mode.

### CROSS-REFERENCES TO RELATED APPLICATIONS

This application claims the benefit of provisional application No. 63/128,378 entitled COOLPACK, filed Dec. 21, 2020. The provisional application is incorporated in its entirety by reference.

### FIELD OF INVENTION

The present invention relates to a device equipped with a body temperature control function. More particularly, the present invention pertains to an article of apparel that provides adjustable cooling or heating functionality to a wearer according to the weather or temperature conditions.

### BACKGROUND OF THE INVENTION

People engaging in activities during inclement conditions, such as extreme weather, often rely on a large amount of specialized insulating equipment to hold in body heat and maintain proper body temperature. For examples, mountain climbers often spend extended periods in extreme cold, at altitude, and may be required to endure snowstorms or other weather events out in the open. Under such conditions, specialized insulated outerwear, hiking boots, hats, gloves, etc., become critical for health and safety.

In some cases, rather than relying solely on passive heat retention/insulation, a heat pack or other heat source may be used to actively generate additional heat. Heat packs may be attached to a body or carried in pockets.

At the opposite extreme, outdoor activity in hot weather during the summer months and in environments such as deserts or grasslands with high exposure to the suns heat and little shade can lead to rapid exhaustion and even heat stroke. These heat impacts are further exacerbated when combined with more rigorous activities, such as hiking or trekking on steep uphill trails, particularly while carrying heavy equipment or devices.

For at least the reasons discussed above, there exists a need for a reliable body temperature management system for use in both extreme cold and extreme heat conditions.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

Embodiments of the present invention comprise a wearable device providing temperature management functionality by cooling or heating the devices surface facing the wearer in order to adjust the wearer's body temperature according to the weather or temperature conditions through application of the Peltier effect.

Thermoelectric cooling uses the Peltier effect to creates a heat flux at a junction of two different types of materials. A Peltier cooler/heater/thermoelectric heat pump is a solid-state active heat pump which uses electricity to transfer heat from one side of the device to the other and the direction of heat transfer can be reversed by reversing the electric current.

### BRIEF DESCRIPTION OF THE DRAWINGS

The present invention is described in detail below with reference to the attached drawing figures, wherein:

FIG. 1 provides a view of two users wearing exemplary embodiments in accordance with the present invention.

FIG. 2 provides a view of a user wearing an exemplary embodiment in accordance with the present invention.

FIG. 3a provides a back view of an exemplary embodiment in accordance with the present invention.

FIG. 3b provides a front view of an exemplary embodiment in accordance with the present invention.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

Embodiments of the present invention comprise a wearable device providing temperature management functionality by cooling or heating the devices surface facing the wearer in order to adjust the wearer's body temperature according to the weather or temperature conditions through application of the Peltier effect.

Thermoelectric cooling uses the Peltier effect to creates a heat flux at a junction of two different types of materials. A Peltier cooler/heater/thermoelectric heat pump is a solid-state active heat pump which uses electricity to transfer heat from one side of the device to the other and the direction of heat transfer can be reversed by reversing the electric current.

As used herein, the terms “Peltier heater”, “Peltier cooler”, “Peltier heat pump”, “solid state refrigerator”, and/or “thermoelectric cooler (TEC)” may be used interchangeably. A Peltier heat pump may be used either for heating or for cooling, though in practice their main application in industry is cooling. Though far less commonly applied to refrigeration than vapor-compression refrigeration, the primary advantages of a Peltier cooler is its lack of moving parts, resulting in very long operational life, and ability for small size and flexibility in shape and design. Additionally, as Peltier coolers use no circulating liquid, they are invulnerability to leaks.

A Peltier cooler can also be used as a thermoelectric generator. When operated as a cooler, a voltage is applied across the device, and as a result, a difference in temperature will build up between the two sides. When operated as a generator, one side of the device is heated to a temperature greater than the other side, and as a result, a difference in voltage will build up between the two sides.

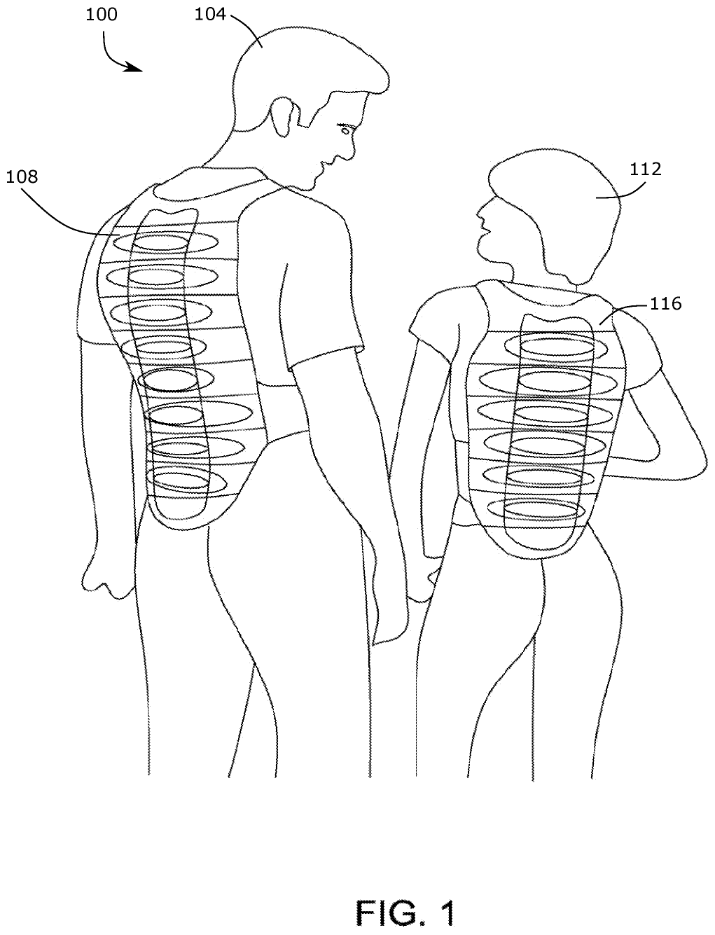

In a first embodiment according to the present invention, a device, using the Peltier cooling effect is created according to FIGS. 1-3. The device has several sections which can independently utilize the Peltier cooling effect. Each section can be controlled independently of the others and can either heat or cool as desired.

FIG. 1 provides a view 100 of two users wearing exemplary embodiments in accordance with the present invention. The first user 104 is wearing a wearable cooling device 108 and the second user 112 is wearing a second cooling device 116. As depicted, the wearable cooling devices 108, 116 are worn on the backs of the users 104, 112.

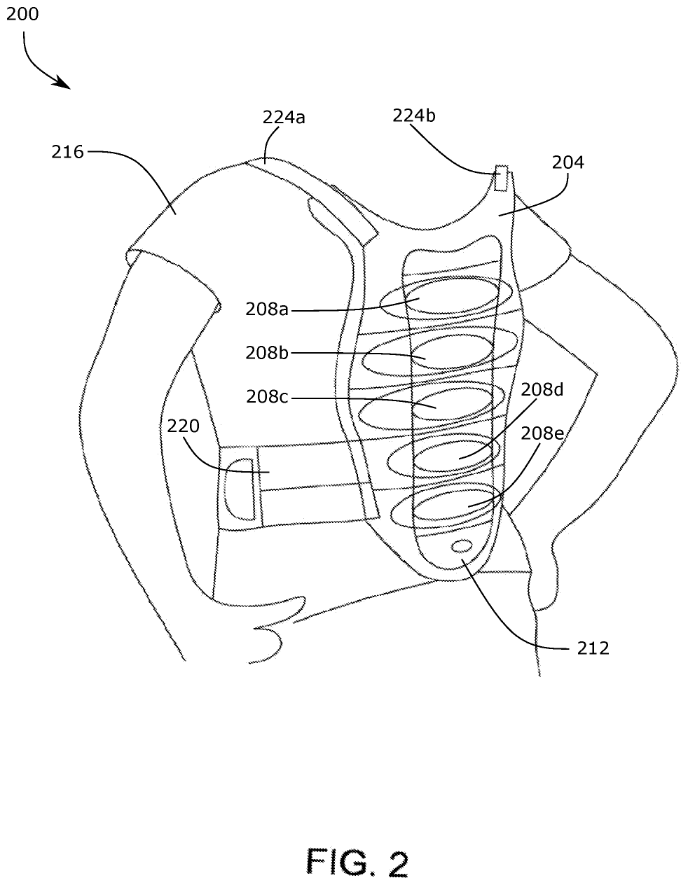

FIG. 2 provides a view 200 of a user wearing an exemplary embodiment in accordance with the present invention. As depicted, wearable device 204 includes 5 segments 208 a-e. In some embodiments, each segment may correspond to a separate heating/cooling panel which is capable of independent heating/cooling. The wearable device 204 includes a battery pack 212 and is secured to the user 216 via waist strap 220 and shoulder straps 224a-b.

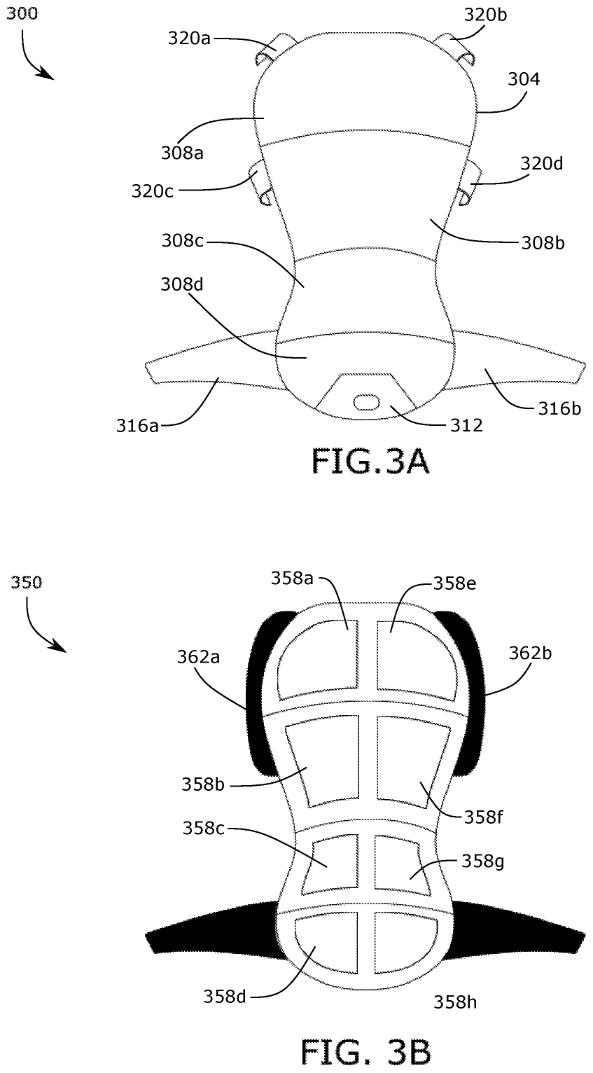

FIG. 3a provides a back view 300 of an exemplary embodiment in accordance with the present invention. As depicted, the wearable device 304 comprises 4 segments 308a-d. The wearable device includes a battery pack 312. Waist strap segments 316a-b that connect at the front of the users to secure the device. Arm strap segments 320a-d secure around the user's shoulders to hold the device to the users back.

FIG. 3b provides a front view 350 of an exemplary embodiment in accordance with the present invention. Wearable device 354 include elements 358a-h, which may be independently heated or cooled. Control pads 362a-b may provide the user with settings control for the wearable device 354.

In some embodiments, the device is powered by a battery located on or in the device. The device's battery may be charged by this connecting to an external power source, such as a standard wall outlet, USB port, or other standard charging mechanism. In some embodiments, the device is powered directly by connecting the device to an external power source. In some embodiments, the battery may be removable and interchangeable, similar to the type of battery used in some cordless power tools. This may allow for easy removal and replacement in the field due the battery being drained or battery failure.

In some embodiments, the device may be wirelessly connected to a smart phone, tablet, computer or other computing device is running a compatible app. Such an app may be used to selectively heat or cool each section of the device. For example, in a device with 8 sections, 4 of them may be providing heat while the remaining 4 may be cooling at the same time. The app on the computing device may display various information, such as battery life, which sections of the device are active, the temperature of each section, and other readings as needed. The temperature of the sections may be determined by a thermocouples or other temperature measuring devices.

In operation, the device may be placed under clothing (as an independent device with shoulder and a belt straps), over clothing, or inserted into a pocket located in a specially designed shirt, jacket or backpack. The device may be able to be plugged in via any standard outlet, USB port, or other standard charging mechanism to recharge the battery. The device may have the hot and cold settings adjusted by an app that you could download on your phone that would also track battery capacity. Embodiments may be able to fit under clothing comfortably without suffering movement impairment.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various figures need be carried out in the specific order described.

What is claimed is:

1. A device comprising:
a device body having an article storage portion contained within the device body;
a shoulder strap suspended from the device body;
a back portion configured to face a back of a wearer,
wherein the back portion is provided with a heat dissipation member that is selectively heated or cooled by the thermoelectric module the device body,
a battery unit for applying power to the thermoelectric module and the thermostat module is provided with a temperature control function, characterized in that the control unit is provided with a switch formed to switch the power on or off, the temperature control mode or the cooling mode or heating mode device.

2. The device of independent claim 1, further comprising a body temperature control function wherein the battery unit is a solar cell unit including a solar cell exposed to an outer surface of the device body.

3. The device of independent claim 1, wherein:
the backrest part of the device body is provided with a backrest plate which is spaced apart from the device body wherein the thermoelectric module and the heat dissipation member are provided. Device having a temperature control function, characterized in that 4 is provided integrally or detachably with the back plate.

4. The device of independent claim 1, further comprising
with a waist belt,
wherein at least one of the shoulder strap and the waist belt includes a thermoelectric module,
wherein the thermoelectric module has a body temperature control function, characterized in that provided with a heat radiation member that is heated or cooled by.

5. The device of independent claim 1, further comprising:
a device body having an article storage portion therein; and
a shoulder strap suspended from the device body,
wherein the back portion facing the back of the wearer of the device body is provided with a planar heating element,
wherein the device body comprises a battery unit for applying power to the planar heating element; and
a power switch for turning on and off the power of the planar heating element.

6. The device of independent claim 1, further comprising:
an operating unit having a temperature control unit; and
wherein the battery unit further comprises a solar cell exposed to the outer surface of the device body.
